(19) United States
(12) Patent Application Publication
(10) Pub. No.: US 2006/0095065 A1
(43) Pub. Date: May 4, 2006

(54) Fluid occluding devices and methods

(76) Inventors: Tetsuaki Tanimura, Acton, MA (US); Christopher Lee, Tewksbury, MA (US); Tetsuya Nakamatsu, Arlington, MA (US); Oudomsith Khoune, Fremont, CA (US); Christopher L. Petersen, Carlisle, MA (US)

Correspondence Address: KIRKPATRICK & LOCKHART NICHOLSON GRAHAM LLP;(FORMERLY KIRKPATRICK & LOCKHART LLP), 75 STATE STREET, BOSTON, MA, 02109-1808, US

(21) Appl. No.: 11/235,692

(22) Filed: Sep. 26, 2005

Related U.S. Application Data
(60) Provisional application No. 60/613,062, filed on Sep. 24, 2004.

(51) Int. Cl. A61M 29/00

(52) U.S. Cl. 606/194

(57) ABSTRACT

In one aspect, the invention relates to a method of occluding blood in a blood vessel during the imaging of a portion of the blood vessel. The method includes the steps of selecting an inflatable element such that diameter of the inflated inflatable element is greater than the diameter of the blood vessel being imaged, introducing the inflatable element into the blood vessel and underinflating the inflatable element such that the vessel wall is not substantially deformed by the inflatable element, the inflatable element substantially occluding the blood vessel to reduce imaging distortion resulting from vessel fluids.

### RELATED APPLICATION

This application claims priority to U.S. Provisional Application 60/613,062 filed on Sep. 24, 2004, the disclosure of which is herein incorporated by reference in its entirety.

### FIELD OF THE INVENTION

The invention relates generally to the field of fluid occluding devices suitable for use in a human or animal. Specifically, the invention relates to catheters, inflatable elements and occluding methods suitable for use with an imaging system such as an Optical Coherence Tomography system.

### BACKGROUND OF THE INVENTION

Optical Coherence Tomography (OCT) is a tomographic optical imaging modality that can produce high resolution (1-15 μm) tomographic images with a depth penetration of 1 to 2 mm in most tissues. OCT is used in medical applications, most notably ophthalmology, but also in oncology and cardiology. In vivo imaging of the walls of coronary vessels has been successfully performed many times by various groups. However, the primary factor limiting OCT's widespread use and acceptance in cardiology relates to wavelength scattering. Specifically, the visible and near infrared wavelengths used by OCT are severely scattered by red blood cells, dramatically reducing the ability to image in the presence of a blood field.

Initially, it was believed that the presence of blood within the vessel would not dramatically affect OCT image quality. However, in vivo studies performed in rabbit aortas indicated that when blood was present, the OCT signal was severely attenuated. This attenuation may be due to either the absorption of hemoglobin or the scattering properties of the red blood cells. Using spectrophotometic analysis of both blood and a hemoglobin-based blood substitute (Oxyglobin), the majority of the attenuation appears to be due to the scattering properties of the erythrocytes. Furthermore, lysing whole blood diminishes the attenuation.

To circumvent this problem, saline flushes have been used to clear the imaging field both in animals and humans. No complications or adverse events were detected using an 8-10 cc flush for imaging human coronary vessels. The main drawback was the limited time window (averaging 2.8 sec) available for imaging when the saline was administered through the guide catheter, the most common and simplest approach. Furthermore, the efficacy of this flush technique is severely limited as the mixing efficiency of the saline is greatly diminished from the time it exits the proximal end of the guide catheter (located at the entrance to the coronary arteries) and the time it reaches the distal imaging location inside the artery.

Another approach to improve in vivo imaging focuses on stopping the flow of blood such that it does not interfere with the imaging system. However, in vivo stoppage of blood flow in coronary arteries is problematic for several reasons. For example, blocking blood flow to the heart muscle for only one minute can cause a heart attack. Additionally, many methods of flow occlusion (e.g. commonplace angioplasty balloon catheters) can cause damage to the arterial walls resulting in both acute and chronic stenosis (vessel narrowing).

Given the improved image data provided by OCT imaging, there exists a need to provide mechanisms to improve in vivo imaging. Specifically, a need exists to remove blood in a region of interest, especially blood near the luminal walls as the detailed investigation of the walls is an important factor in arterial disease diagnosis.

### SUMMARY OF THE INVENTION

Within human or animal vessels and lumens, it is desirable to achieve OCT imaging in a short, but useful time period (˜30-40 seconds). It is also important to minimize any risk of harm to the coronary arteries. The aspects and embodiments of the invention disclosed herein are designed, in part, to improve imaging and address the associated problems with imaging in a vessel of interest. As such, various balloon catheter designs and methods that can achieve the desired result for cardiac OCT and other in vivo OCT applications are described in more detail below.

A volume controlled balloon or other inflatable element is one aspect of the invention. The balloon is volume controlled in the sense that it is underinflated when operating as an occluder. The balloon can be operated at a fixed, low pressure in arteries or other vessels up to the nominal fully inflated balloon size. Thus, high pressure is not required to inflate the balloon. In fact, high pressure balloon inflation is specifically avoided in these embodiments. The choice of low pressure volume controlled balloons prevents the vessel from being expanded (even temporarily) and reduces the risk of damage to the vessel wall.

While in an underinflated state, the inflatable element typically has folds in its surface. These folds are indicative of its low pressure and partially inflated state. As such, the inflatable element does not substantially transform or otherwise distend the walls of the vessel within which it is disposed. In one embodiment, the folds facilitate occluding vessel fluids to enhance optical scanning via a probe element in combination with an imaging system, such as an OCT system. Additionally, the remaining folds eliminate the need for applying pressure to stretch the balloon material, thereby minimizing the pressure.

Blood vessel expansion by high pressure balloon angioplasty has severe acute and chronic effects. The acute effects are primarily spasms which constricts the artery, shutting down blood flow to critical areas of the heart. The chronic effects include significant restenosis of the artery, often worse than any existing stenotic condition. As a result, safety provisions must be in place during a balloon-based occlusive approach. In particular, this is achieved by selecting a balloon that is deliberately oversized for the target vessel. Furthermore, the balloon is not highly elastic such that forceful stretching of the balloon material is not required to occlude the vessel.

Although oversized, the balloons are not fully inflated when introduced into a given lumen, but rather selectively inflated to occlude, but not expand or deform the artery or other vessel of interest. Since imaging occurs distal to the balloon, rather than within the balloon as in angioplasty balloons, folds and wrinkles in the under-expanded balloon do not degrade image quality. Furthermore, since a flush is also provided distal to the balloon, any small blood leakage through a fold is rapidly diluted.

In one aspect, the invention relates to a method of occluding blood in a blood vessel during the imaging of a portion of the blood vessel. The method includes the steps of selecting an inflatable element such that diameter of the inflated inflatable element is greater than the diameter of the blood vessel being imaged, introducing the inflatable element into the blood vessel and underinflating the inflatable element such that the vessel wall is not substantially deformed by the inflatable element, the inflatable element substantially occluding the blood vessel to reduce imaging distortion resulting from vessel fluids. The inflatable element includes an expandable membrane in one embodiment of the method. The surface of the expandable membrane can include folds when contacting the vessel wall. In one embodiment, the inflatable element includes a non-compliant or semi-compliant balloon. Also, the method can further include the step of flushing the blood vessel with a fluid in a direction retrograde to a direction of normal blood flow.

In another aspect, the invention relates to a fluid occluding device. The device includes an inflatable catheter and an imaging system to improve imaging quality by substantially blocking intra-vessel fluids. In turn, the inflatable catheter includes a balloon portion having a vessel contacting surface such that the balloon portion is oversized in relation to the vessel of interest. In one embodiment, the balloon portion has a diameter that ranges from about 2 mm to about 4 mm. The inflation pressure of balloon portion can range from about 150 mbar to about 750 mbar in various embodiments. Also, in one embodiment the vessel contacting surface includes folds when in an underinflated state. The inflatable element can also include a hydrophobic coating applied to the vessel contacting surface.

In yet another aspect, the invention relates to a balloon catheter system. The system includes a balloon connected to an inflation lumen, and a combined flushing and imaging lumen extending distal to the balloon, at least one coaxial exit aperture to the imaging lumen, and a plurality of exit apertures along the imaging lumen, wherein the balloon operating pressures are substantially below one atmosphere. The plurality of exit apertures can direct the flush flow at an angle retrograde to the normal blood flow.

In one embodiment, the portion of the imaging lumen distal to the balloon is adapted to be atraumatic to blood vessels. At least some of the exit apertures are orientated such that ejected flush solution is substantially retrograde to normal blood flow in one embodiment. The flush lumen can include several exit apertures arranged both longitudinally and circumferentially around the lumen to increase the effectiveness of the flush solution.

Also, the balloon is adapted to keep the combined flushing and imaging lumen substantially optically clear at least in a vessel segment proximate to the balloon in one embodiment. The exit apertures along the imaging lumen can be adapted to direct flush solution against the wall of the inflated balloon to further increase the turbulence of the flush solution to improve mixing with and clearing of residual arterial blood. In one embodiment, the flush imaging lumen is adapted to provide stabilizing support for an imaging optical fiber. An OCT channel can be included in at least one of the balloon, catheter, or both.

In another aspect, the invention relates to a method of imaging a portion of a vessel having a vessel wall. The method includes the steps of introducing an inflatable element having a volume within the vessel, imaging the vessel wall while fluid is flowing through the vessel, increasing the volume of the inflatable element incrementally such that image distortion effects caused by the fluid are substantially reduced without distorting the vessel. In one embodiment of the method, the inflatable element is non-compliant. The method further includes the step of controlling the volume of the inflatable element such that trauma to the vessel wall is substantially reduced in one embodiment. The portion of the vessel is imaged using an optical coherence tomography probe disposed in the vessel in one embodiment. In one embodiment of the method, the inflatable element includes a hydrophobic coating.

Various catheters and catheter elements are also disclosed herein that represent additional aspects of the invention.

It should be understood that the terms “a,” “an,” and “the” mean “one or more,” unless expressly specified otherwise.

The foregoing, and other features and advantages of the invention, as well as the invention itself, will be more fully understood from the description, drawings, and claims which follow.

### BRIEF DESCRIPTION OF THE FIGURES

Reference to the figures herein is intended to provide a better understanding of the methods and apparatus of the invention but are not intended to limit the scope of the invention to the specifically depicted embodiments. The drawings are not necessarily to scale, emphasis instead being placed upon illustrating the principles of the invention. Like reference characters in the respective figures typically indicate corresponding parts.

FIG. 1A is a schematic diagram depicting a catheter system in accordance with an illustrative embodiment of the invention.

FIGS. 1B and 1C are schematic diagrams depicting catheter system in accordance with an illustrative embodiment of the invention.

FIG. 2 is a schematic diagram depicting a fiber-optic imaging subsystem in accordance with an illustrative embodiment of the invention.

FIG. 3 is a plot depicting the diameter versus pressure relationship for a balloon in accordance with an illustrative embodiment of the invention.

FIGS. 4 and 5 are schematic diagrams depicting fluid occluding and flushing components of an inflated balloon in accordance with an illustrative embodiment of the invention.

FIGS. 6A and 6B are schematic diagrams depicting imaging data captured with a balloon catheter during an in vivo animal trial in accordance with an illustrative embodiment of the invention.

FIGS. 6C and 6D are images depicting the imaging data represented in FIGS. 6A and 6B in accordance with an illustrative embodiment of the invention.

FIG. 6E is an image of an inflatable element inflated at low pressure having surface folds in accordance with an illustrative embodiment o the invention.

FIG. 7 is a block diagram depicting a method of adjusting the volume of an inflatable element to improve imaging of a vessel of interest in accordance with an illustrative embodiment of the invention.

The claimed invention will be more completely understood through the following detailed description, which should be read in conjunction with the attached drawings. In this description, like numbers refer to similar elements within various embodiments of the present invention.

### DETAILED DESCRIPTION

The following description refers to the accompanying drawings that illustrate certain embodiments of the present invention. Other embodiments are possible and modifications may be made to the embodiments without departing from the spirit and scope of the invention. Therefore, the following detailed description is not meant to limit the present invention. Rather, the scope of the present invention is defined by the appended claims.

It should be understood that the order of the steps of the methods of the invention is immaterial so long as the invention remains operable. Moreover, two or more steps may be conducted simultaneously or in a different order than recited herein unless otherwise specified.

The balloons typically used in cardiac applications and other in vivo imaging systems are compliant balloons. A compliant balloon is analogous to a toy balloon. For a compliant balloon, greater pressure results in a greater increase in the size of the balloon. Also, when used in vivo, compliant angioplasty balloons are fully inflated. Furthermore, compliant balloons tend to be rigid and designed with a high inflation pressure compared with a non-compliant balloon. As a result, the balloons can cause damage to the arterial wall by distending or remodeling the vessel. In vivo balloon bursting is also a risk associated with compliant balloons when fully inflated under high pressure. In contrast, a volume limited balloon inflates to contact the vessel diameter when subjected to a low inflation pressure.

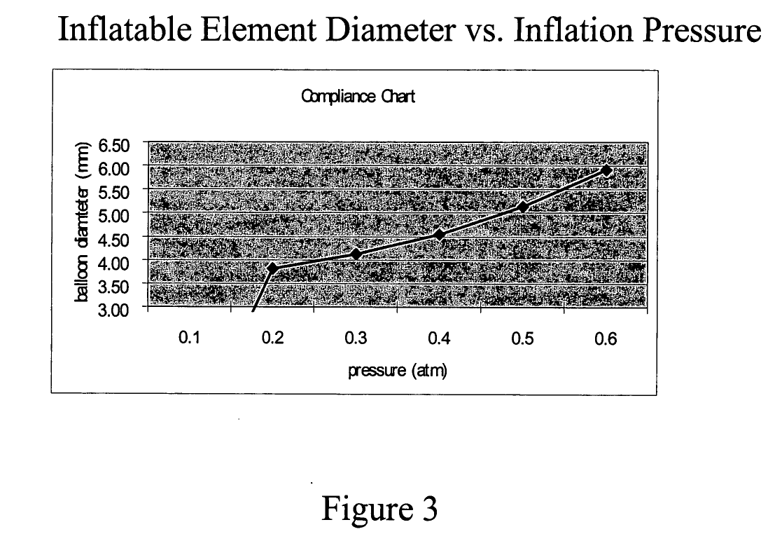

In contrast to various prior art approaches that use fully inflated balloons, one aspect of the invention relates to using oversized inflatable elements, in coronary and other human/animal imaging applications. However, the oversized inflatable elements are maintained in an underinflated state when used in vivo. Non-compliant and semi-compliant balloons can be used in different embodiments subject to the size selection and inflation levels described herein. Additional details relating to non-compliant and semi-compliant balloons are described in more detail below with respect to FIG. 3.

The balloons used in a given imaging application are oversized such that when fully inflated at least one dimension, such as a balloon diameter, is larger than a corresponding dimension in a vessel of interest. Thus, in one embodiment for the oversized balloons or other suitable inflatable elements, the balloon nominal diameter is actually larger than the vessel diameter. However, once the appropriate size is selected relative to a vessel of interest, when inflated in the vessel, the pressure and/or volume of the balloon is regulated such that the balloon remains underinflated while imaging of the vessel takes place.

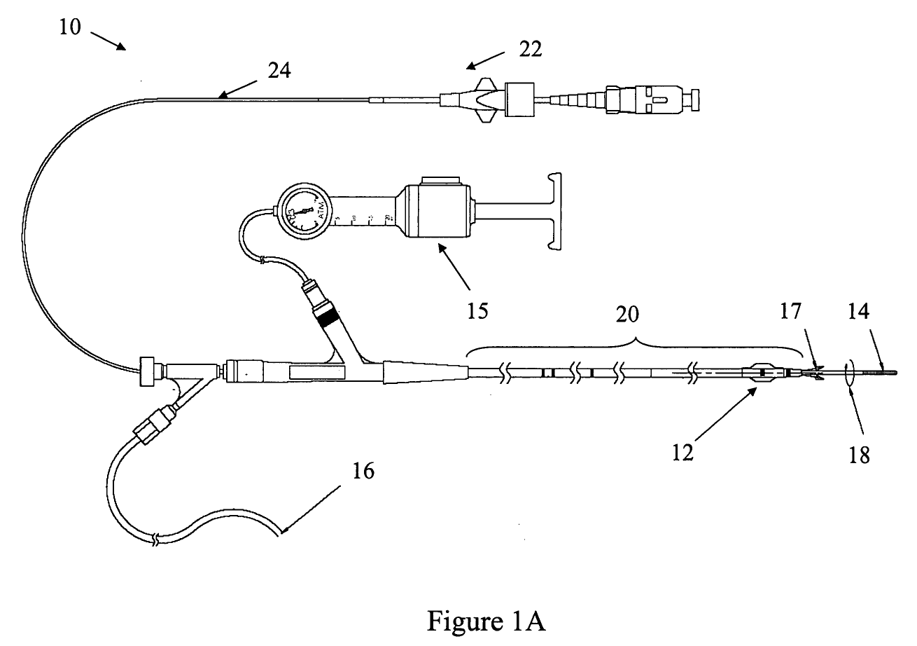

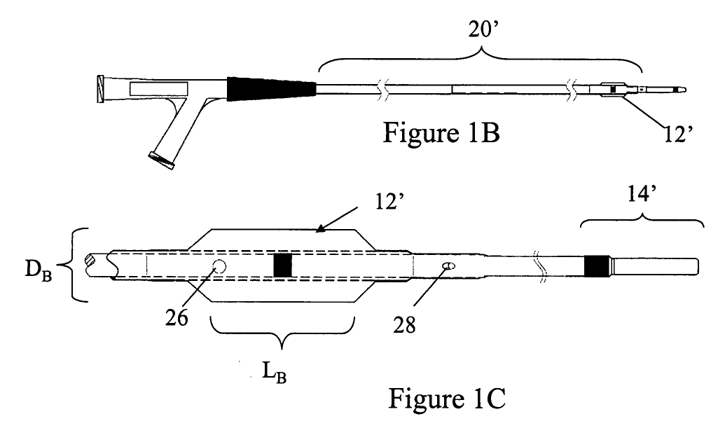

Accordingly, although somewhat counterintuitive, using a deliberately oversized balloon is advantageous. This follows because the risk of damage to vessel walls is reduced. In one embodiment, the additional flexible material used to form the balloon provides additional occluding benefits by remaining loose in the vessel when the balloon is in its underinflated state. FIG. 1A depicts features of a balloon catheter system 10 suitable for performing imaging scans of a vessel of interest. The inflatable element 12 shown is used to occlude vessel fluids to reduce optical distortion when imaging a portion of a vessel wall. As discussed above, the inflatable element is typically a non-compliant balloon that has a fully inflated diameter that exceeds the diameter of the vessel of interest. The exemplary balloon catheter system 10 also includes an imaging probe 14 containing a rotating optical fiber portion, and an inflation/deflation device 15 with pressure gauge for inflating and deflating the inflatable element 12. The inflatable element 12 is typically inflated using a liquid, such as saline or a solution of saline in a radio-opaque dye. Also, the catheter material (not shown) surrounding the tip of the probe 14 is typically chosen to transmit OCT wavelengths emitted and received by the optical fiber portion and to introduce minimal scattering.

The system 10 also includes a tube 16 for providing flush solution, such as saline, an exemplary flush port 17 is also shown (shown by arrow). The optical fiber portion of the probe 14 is rotatable as shown by arrow 18. The catheter portion 20 shown contains two or more lumens. In this embodiment, one inflation lumen is in fluid communication with the inflation element 12. As such, the single optical fiber and flushing system coexist in another lumen. A fiber optic imaging system, such as an OCT system, incorporating interferometric measuring components can also be attached to the system via the optical fiber imaging subsystem 22. The subsystem 22 includes a coated optical fiber 24 that is in optical communication with the probe 14.

FIG. 1B depicts various alternative embodiments of portions of the system 10 shown in FIG. 1A. In turn, FIG. 1C provides additional details relating to the inflatable element. Specifically, a catheter portion 20′ and an inflatable element 12′ are shown. The inflatable element 12′ includes an inflation port 26. The inflation port 26 is adapted to receive an inflating fluid, such as saline, to regulate the volume of the inflatable element 12′. A flushing port 28 is also shown as part of the catheter portion 20′. The probe portion 14′ contains a rotable optical fiber that shares a lumen connected to the flushing port 28 in one embodiment.

FIG. 1C depicts an exemplary embodiment of an inflatable element 12′. In one embodiment of the invention, the balloon system is optimized for OCT imaging in blood-filled lumens, such as for example coronary arteries. The inflatable element 12′ is typically selected to have substantially low inflation pressure, to avoid any damage from expansion of the arterial wall. The inflation pressure will only be slightly above normal arterial pressures (typically in the range of 140 mm Hg) in one embodiment. One embodiment of the invention is constructed using an inflatable element 12′ that includes an outer membrane that is attached to a portion of the catheter.

The dimensions of the inflatable element 12′ are chosen to be compatible with a variety of lumen sizes, up to a maximum size wherein treatment or diagnosis is not normally a concern (e.g. above 4 mm in the coronary system). As shown, the length of the balloon LB and the balloon diameter D_{B} can vary according to the size of the vessel of interest and the location of the vessel in the body. In general, the diameter or cross-section of the inflatable element is larger than that of the target vessels. As a result, in various implementations the inflatable element is operated based on volume controlled rather than pressure controlled restrictions. The balloon diameter D_{B} can range from about 2 mm to about 7 mm in one embodiment. Similarly, the balloon length can range from about 2 mm to about 10 mm in one embodiment. Exemplary dimensions relating to a specific catheter and inflatable element embodiments are described herein. However, variations from these dimensions and other suitable balloon and catheter dimensions as known in the art are also suitable for certain embodiments.

In one embodiment, the membrane of the balloon includes a polyurethane material. A polyethylene terephthalate (PET) or nylon material is used to make the balloon's surface material in one embodiment. In one inflatable element embodiment a hydrophobic coating is applied on the element's surface. The application of the coating can improve the occluding properties of a under-inflated balloon by preventing blood flow through any small openings in folds of the balloons. The hydrophobic coating can include PTFE (polytetrafluoroethylene) or silicone-based compounds. In general, the material used to fabricate the inflatable elements disclosed herein is selected to provide flexibility in bending such that the material can be unfolded at low pressures. Thus, the material is sufficiently flexible that increased pressure is not required to initially unfold the material in vivo. Therefore, polyurethane and Pebax materials may can be used in various embodiments. Balloon wall thicknesses in the range of 0.0005″ can also enhance flexibility and also have the benefit of minimizing catheter dimensions.

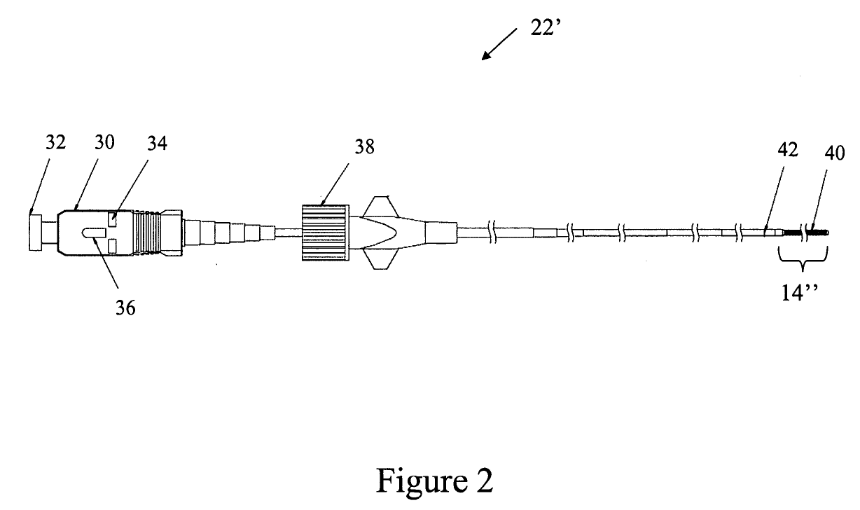

As shown in FIG. 2 additional details relating to an optical fiber imaging subsystem 22′ are shown. The subsystem 22′ includes a standard fiber optic connector (type SC) 30, a protective cap 32, engagement points 34, 36 for the mating socket, a standard Luer adapter 38 which provide a mounting interface for the imaging lumen and/or an optical fiber connected to an OCT imaging system. Different portions of the system can rotate for example, in the embodiment shown the inner (rotating) sheath 40 and the outer (non-rotating) sheath 42 are shown. The inner rotating sheath 40 of the imaging probe 14″ includes an optical fiber portion. In one embodiment, the optical fiber portion is surrounded by a viscous damping fluid to substantially reduce non-uniform rotational distortion.

FIG. 3 is a plot showing the nominal diameter versus pressure relationship for a particular semi-compliant inflatable element. The relationship depicted in the plot illustrates the volume controlled aspect of an exemplary semi-compliant balloon. The plot indicates that the balloon diameter increases rapidly without a dependence on pressure until the nominal 4 mm diameter is reached. After the 4 mm diameter point, the balloon exhibits a substantially linear dependence of diameter with pressure. At the balloon's maximum volume level (6 mm diameter), increasing pressure does not increase volume. Since the balloon embodiment shown has a nominal diameter range of approximately 4 mm, it can be used in vessels of interest ranging from about 1.5 mm to about 4 mm. Once inserted in the vessel of interest the diameter/pressure relationship can be used to ensure that the balloon remains underinflated while still providing an occluding effect as a result of the volume occupied in the vessel of interest. Accordingly, a low-pressure balloon that is not completely expanded to its final size (i.e. underinflated), but does fully or substantially expand to the vessel size is one aspect of the claimed invention.

Thus, semi-compliant and non-compliant balloons can be used in various embodiments. The discontinuity depicted in FIG. 3 wherein the gradual lower pressure filling of the balloon changes to a substantially linear pressure to volume relationship is indicative of a semi-compliant balloon. Therefore, after the nominal size is reached at or about the discontinuity; increases in pressure increase the volume, but a relatively large pressure change is needed for a given volume change in a semi-compliant balloon. For an ideal non-compliant balloon, the pressure/volume curve after the discontinuity typically remains flat at the nominal balloon diameter.

In another exemplary embodiment, the nominal balloon diameter, that is the diameter the balloon reaches at its normal operating pressure (˜300 to 500 millibar (˜0.2961 to 0.4935 atm) would be approximately 4 millimeters. Thus, in an open space, open air environment, if 300 millibar pressure is introduced in the balloon, a nominal balloon diameter of 4 millimeters results. In the in vivo indications for use, the target of 4 millimeter balloon diameter for arteries 4 millimeters or smaller is appropriate. Therefore, in one embodiment, at 0.3 bar, the balloon expands to the size of the vessel, substantially or completely occluding the vessel. Accordingly, for any vessel diameter up to 4 millimeters, the same pressure will cause the selected balloon to inflate to the size of the vessel. This differs from the compliant balloons discussed above.

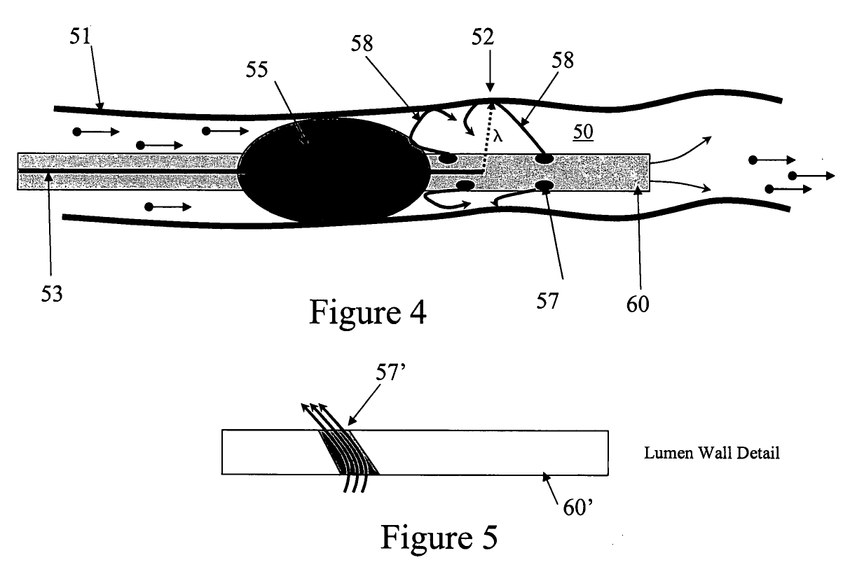

FIGS. 4 and 5 provide details relating to the placement of the inflatable element and the use of directed retrograde flushes in accordance with the invention. Prior to considering these figures in more detail, consideration of the relevant problems in the prior art is informative. In the prior art, to obtain OCT images using a combination of an imaging probe and an angioplasty balloon, a conventional guide wire is inserted into the target artery, providing a path for diagnostic and therapeutic devices. The angioplasty balloon catheter is inserted over the guide wire and located in the desired region (constriction, suspect lesion, etc). In this case, imaging is performed through the balloon, as there is no provision for clearing the blood distal to the balloon. There are several distinct disadvantages to this technique. The first disadvantage is that the balloon is rigid and must be specifically sized for the target vessel. Furthermore, as a second disadvantage the balloon must be inflated to a high pressure (6-12 atmospheres). This high pressure and the resulting force on the artery can cause significant injury to the arterial wall. A third disadvantage with this approach occurs when blood is trapped between the balloon and irregular lumen geometries. From an imaging standpoint, dense trapped blood and soft arterial tissue are very difficult to distinguish. The devices depicted in FIGS. 4 and 5 overcome these disadvantages.

In contrast, the imaging and fluid occluding techniques disclosed herein use a different approach that addresses the problems associated with various prior art approaches. One exemplary fluid occluding and imaging technique and system portion are depicted in FIG. 4. In FIG. 4 a vessel of interest 50 having a vessel wall 51 is depicted. The direction of blood flow is left to right as indicated by the arrows in the vessel 50. The portion of the vessel wall being imaged is shown at point 52. The vessel of interest may be an artery and the overall imaging system may be used to determine if the artery contains plaques or other indicia of current or future cardiac system problems. As shown, distal means deeper into the vessel of interest, in embodiment show, further way from the flush ports on the catheter portion described below, to the right of the inflatable element.

A balloon catheter portion that includes a rotatable optical fiber imaging portion 53 for emitting and receiving light λ, an inflatable element 55, a plurality of flush ports 57 with flow lines 58 depicting a retrograde flush, and a combined imaging and flushing lumen 60 are shown. A separate inflating lumen disposed within the combined lumen 60 suitable for partially inflating the inflatable element is typically part of the overall catheter. In some embodiments, the two lumens are disposed in parallel or arranged concentrically.

As discussed above, the presence of a blood field detrimentally affects the ability to image a portion of a vessel of interest. The use of a non-compliant oversized underinflated balloon in combination with a retrograde flushing system solves the problem of blood distorting an image of a portion of the vessel of interest. As shown by the flow lines 58 emanating from the flush ports 57, the flushing system provides a retrograde flush distal to the inflatable element 55 in order to clear the blood field for imaging at point 52. The flush is retrograde as it is directed opposite the direction of blood flow. As a result, substantial portions of the flush are ejected in directions non-coaxial with the catheter to provide better mixing and removal of blood near the vessel walls.

In addition, as shown in FIG. 5, a flush that is ejected such that it impinges on the distal wall of the inflated balloon is effective at mixing with and clearing blood as it forms a turbulent zone. Thus, one aspect of the invention is directed to the arrangement of the flush ports 57′ of the balloon catheter described herein such that they are angled to direct a flushing solutions backwards toward the inflatable element 55. The arrangement of angled flush ports facilitates safe and effective blood clearing via a controlled flush distal to the balloon 55. As a result, imaging that would otherwise be distorted by the presence of blood is now possible using the approaches depicted above and in FIGS. 4 and 5.

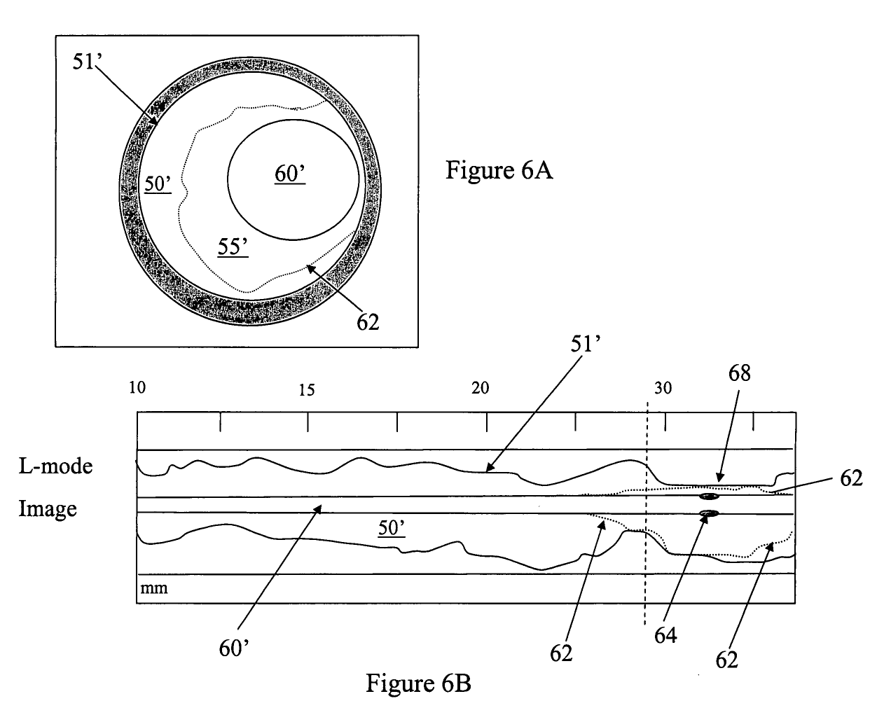

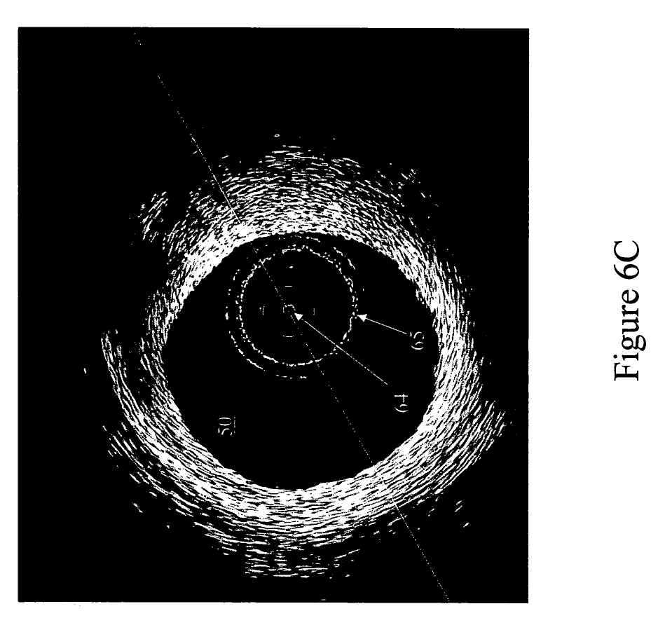

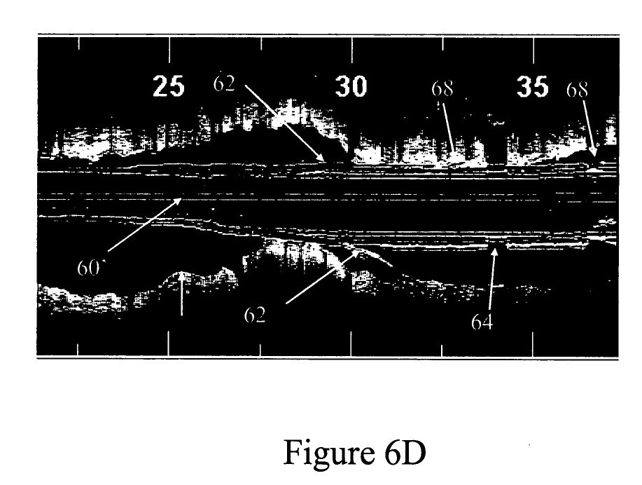

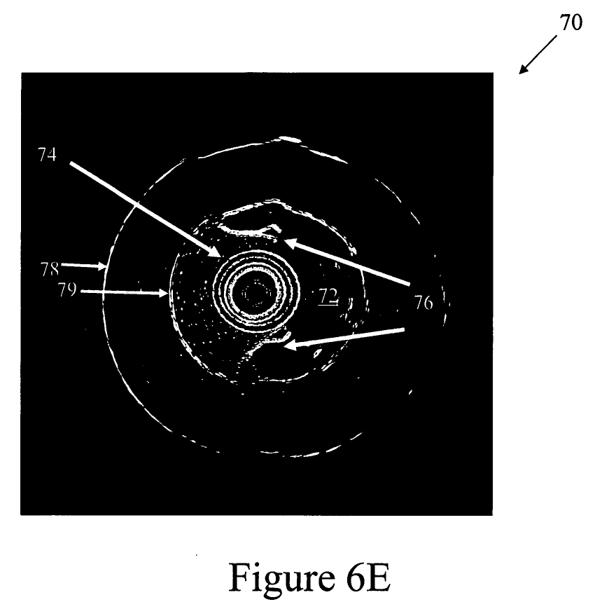

FIGS. 6A and 6B depict representations of imaging data captured using an OCT system incorporating a balloon catheter as described herein during an in vivo animal trial. The actual image data that was obtained during the trial is illustrated in FIGS. 6C and 6D. In particular, FIG. 6A is a schematic diagram of the image shown in FIG. 6C.

The image in FIGS. 6A and 6C is a cross section of the coronary artery at the location shown by the vertical dotted line in the lower image. The image in FIGS. 6B and 6D depicts the entire image set during a series of sequential scans wherein the imaging probe (not shown) was moved along the length of a vessel of interest. A vessel of interest 50′ having a vessel wall 51′ was imaged using a balloon catheter having a catheter portion 60′ and a balloon 55′ having an external balloon wall 62. The catheter portion 60′ also includes inflation ports 64. Specifically, the catheter portion 60′ and image probe are extended distally. Next the probe was pulled back at a controlled rate in the artery while imaging with the balloon inflated and flush solution being applied as describe above. This provides a longitudinal cross-sectional survey of the arterial lumen, referred to as an L-mode image. The L-mode image schematically represented in FIG. 6B and illustrated pictorially in FIG. 6D is a lengthwise optical cross-section of an artery as captured by an optical probe during repeat balloon inflation/deflation cycles and probe movement along the artery. The cross-section of the balloon wall 62 can be seen in the right portion of FIGS. 6B and 6D. The optical fiber portion 64 and imaging/flushing lumen 65 is seen in the center of the lumen in FIG. 6C.

Several observations are discernable from the set of OCT data that is represented in FIGS. 6A-6D. The data and images depicted in the figures was obtained using a configuration similar to that shown in FIG. 4. As a result, the vessel 50′ is substantially clear from any residual blood as the vessel wall is clearly shown. In addition, the balloon wall 62 conforms to the vessel wall's 51 shape without distorting or remodeling the wall 51. As depicted the balloon's outer membrane portion or wall 62 has a number of folds or surface irregularities. These folds and irregularities are present because although the balloon has successfully occluded blood flow, the balloon is in an underinflated state. However, since the balloon 55′ was oversized with respect to the vessel of interest 50, occlusion is possible in an underinflated state. As such, the details of the vessel image shown in the FIG. 6D demonstrates the level of imaging capable when using the inflatable elements, catheters, and an OCT system as disclosed herein. Although the contour of the balloon wall 62 tracks the vessel wall 51 at the bottom of the image, the balloon wall 62 does not push or distend the lumen wall at the top portions 68 as shown in FIGS. 6B and 6D. Therefore, the techniques and devices disclosed herein also provide safety advantages over various prior art approaches.

FIG. 6E is an image 70 of an inflatable element inflated at low pressure (0.15 bar) in a tube 72 less than the nominal diameter of the inflatable element. A balloon catheter with an optical probe is present in the tube 72. The folds 76 of the balloon wall are shown relative to the tube walls 78, 79. As shown in FIGS. 6B, 6D and 6E surface irregularities, such as the folds 76 shown, typically exist in the inflatable elements as a result of the underinflation of the balloon. FIG. 6E further demonstrates the level of detail possible as the folds 76 of the balloon can be clearly seen in the image. Thus, in vivo use of the balloons disclosed herein provides beneficial results when used as occluders.

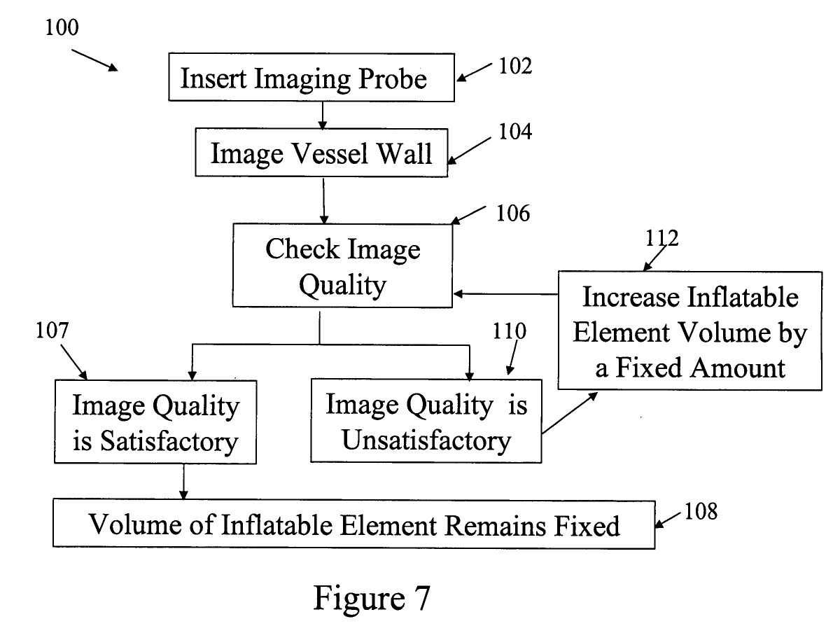

The invention also relates to methods for improving imaging of vessel of interest by reducing optical distortions associated with the obscuring effects of vessel fluids. A method of reducing the impact of vessel fluids is described in FIG. 8. The steps of the method are typically used in combination with an apparatus embodiment suitable for use in vivo as shown in FIG. 5. The method 100 shown describes monitoring the image quality generated by an imaging system, such as an optical coherence tomography system and adjusting an inflatable element until image quality improves to a satisfactory level. Retrograde flushes can also be used with the method 100. Specific metrics, such as existing image quality levels from a non-blood containing environment, can be used to control when image quality is satisfactory. Alternatively, the technician conducting the imaging process can specify certain parameters or otherwise rely on the images they are viewing to control the balloon volume. Typically, the inflatable element used is a non-compliant balloon. As a result, over inflation and/or damage to the vessel of interest is not possible. Pressure versus volume plots such as depicted in FIG. 3 can also be used to calibrate the balloon inflation control system outlined in FIG. 7.

The method 100 depicted in FIG. 7 includes the step of inserting an imaging probe (Step 102) in a vessel of interest. Once inserted and placed at a region of interest in the vessel, the imaging process, typically an OCT data capture, is started (Step 104). The next step is to check the image quality (Step 106), if the image quality is satisfactory (Step 107), the volume of the inflatable element remains fixed (Step 108). However, if during the step of checking the image quality (Step 106), the image quality is unsatisfactory (Step 110), the next step is to increase the inflatable element volume by a fixed amount (Step 112). After the volume increase, the image quality check is repeated (Step 106). As a result, when the image quality is at the desired level, the operator ceases to increase the volume and the image data collection continues with improved image quality. In one embodiment, in step 112 is replaced a pressure increasing step wherein the pressure in the balloon is increased by a set amount. In one embodiment the imaging fiber can be retracted inside the (transparent) balloon to titrate contact with the luminal surface as the balloon inflates in order to determine balloon wall placement relative to the vessel wall of interest.

In addition, the catheter must also fulfill the standard safety requirements. It is desirable that the catheter be easy to use. In one embodiment of the invention, the balloon or other suitable inflatable element is constructed such that when inflated, a substantially clear catheter lumen is present. In turn, the substantially clear lumen facilitates OCT system operation and vessel imaging.

The systems, methods, and devices disclosed herein provide many advantages over the prior art. The present invention allows for independent movement of the single imaging fiber. By using a single optical fiber, an imaging bundle of multiple fibers is avoided. As a result, the overall catheter system is smaller. Furthermore, the optical fiber is not permanently bonded within the catheter, such that any scanning of the interior luminal surface does not require movement of the entire catheter which is a potential safety risk to the lining of the vessel present in some prior art embodiments.

The flushing embodiments disclosed herein allow for flushing sufficient to provide scanning times that are sufficient for a survey of the vessel interest. Also, optimizing the fluid flush direction for effective blood clearing is not described in various prior art approaches. As mentioned previously, it is known that a coaxial flush does not adequately remove blood adjacent to the vessel walls.

Also, in some prior art embodiments, the intended imaging area is directly under the balloon. It is known that trapped blood between the balloon and the vessel wall leads to image artifacts. Lastly, the inflated high-pressure balloon will distort the anatomy being visualized. In fact, the intent of the some prior art balloons is to visualize angioplasty (deliberate vessel remodeling) during the procedure. In the present disclosure, all forms of angioplasty remodeling are avoided.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method of occluding blood in a blood vessel, having a vessel wall, during imaging of a portion of the blood vessel, comprising the steps of:
selecting an inflatable element such that diameter of the inflated inflatable element is greater than the diameter of the blood vessel being imaged;
introducing the inflatable element into the blood vessel; and
underinflating the inflatable element such that the vessel wall is not substantially deformed by the inflatable element, the inflatable element substantially occluding the blood vessel to reduce imaging distortion resulting from vessel fluids.

2. The method of claim 1 wherein the inflatable element comprises an expandable membrane.

3. The method of claim 1 wherein portions of the surface of the expandable membrane comprises folds when contacting the vessel wall.

4. The method of claim 1 wherein the inflatable element comprises a balloon.

5. The method of claim 1 further comprising the step of flushing the blood vessel with a fluid in a direction retrograde to a direction of normal blood flow.

6. A fluid occluding device, the device comprising:
an inflatable catheter and an imaging system to improve imaging quality by substantially blocking intra-vessel fluids,
the inflatable catheter comprising a balloon portion having a vessel contacting surface, wherein the balloon portion is oversized in relation to the vessel of interest.

7. The fluid occluding device of claim 6 wherein the balloon portion has a diameter that ranges from about 2 mm to about 4 mm.

8. The fluid occluding device of claim 6 wherein the inflation pressure of the balloon portion ranges from about 150 mbar to about 750 mbar.

9. The fluid occluding device of claim 6 wherein the vessel contacting surface comprises folds when in an underinflated state.

10. The fluid occluding device of claim 6 wherein the inflatable element comprises a hydrophobic coating applied to the vessel contacting surface.

11. A balloon catheter system, the system comprising:
a balloon connected to an inflation lumen, and
a combined flushing and imaging lumen extending distal to the balloon,
at least one coaxial exit aperture to the imaging lumen, and
a plurality of exit apertures along the imaging lumen, wherein the balloon operating pressures are substantially below 1 atmosphere.

12. The balloon catheter system of claim 11 wherein the plurality of exit apertures direct the flush flow at an angle retrograde to the normal blood flow.

13. The balloon catheter system of claim 11 wherein the portion of the imaging lumen distal to the balloon is adapted to be atraumatic to blood vessels.

14. The balloon catheter system of claim 11 wherein at least some of the exit apertures are orientated such that ejected flush solution is substantially retrograde to normal blood flow.

15. The balloon catheter system of claim 11 wherein the flush imaging lumen has several exit apertures arranged both longitudinally and circumferentially around the lumen to increase the effectiveness of the flush solution.

16. The balloon catheter system of claim 11 wherein the balloon is adapted to keep the combined flushing and imaging lumen substantially optically clear at least in a vessel segment proximate to the balloon.

17. The balloon catheter system of claim 11 wherein the exit apertures along the imaging lumen are adapted to direct flush solution against the wall of the inflated balloon to further increase the turbulence of the flush solution to improve mixing with and clearing of residual arterial blood.

18. The balloon catheter system of claim 11 wherein the flush imaging lumen provides stabilizing support for an imaging optical fiber.

19. The balloon catheter system of claim 11 wherein an OCT channel is included in at least one of the balloon, catheter, or both.

20. A method of imaging a portion of a vessel having a vessel wall, the method comprising the steps of:
introducing an inflatable element having a volume within the vessel;
imaging the vessel wall while fluid is flowing through the vessel;
increasing the volume of the inflatable element incrementally such that image distortion effects caused by the fluid are substantially reduced without distorting the vessel.

21. The method of claim 19 wherein the inflatable element is non-compliant or semi-compliant.

22. The method of claim 19 further comprising the step of controlling the volume of the inflatable element such that trauma to the vessel wall is substantially reduced.

23. The method of claim 19 wherein the portion of the vessel is imaged using an optical coherence tomography probe disposed in the vessel.

24. The method of claim 19 wherein the inflatable element comprises a hydrophobic coating.
